(19) United States
(12) Patent Application Publication
(10) Pub. No.: US 2004/0202631 A1
(43) Pub. Date: Oct. 14, 2004

(54) Hair styling method

(76) Inventors: Yoshihiro Hirata, Kyoto City (JP); Yuko Yamashita, Kyoto-city (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/169,588

(22) PCT Filed: Jan. 5, 2001
(86) PCT No.: PCT/JP01/00017

(30) Foreign Application Priority Data
Jan. 7, 2000 (JP) 2000/1315

(51) Int. Cl. A61K 7/06; G06K 9/00

(52) U.S. Cl. 424/70.1; 382/128

(57) ABSTRACT

By styling hair based on a design-model paper in which a hair design according to a request made by a customer is digitized or coded, a method for designing a hair style and a method for styling hair, which can actualize the customer's request for a hair style accurately as compared with conventional hair styling methods used in hairstyling and hairdressing trades, and at the same time, which can reproduce the hair design without fail, and a design model used for designing a hair style and styling hair using the aforesaid methods, are provided.

In these methods, by entering numerical values and/or codes corresponding to a hair design requested by a customer in each of multiple regions which are created by dividing a developed view of a human-head shape, a hair style can be designed based on the numerical values, and cutting, permanent-waving or coloring hair can be done based on this design.

### TECHNICAL FIELD

The present invention relates to a method for designing hair styling including hair cutting, giving a permanent-wave and hair coloring.

The present invention relates to a method for entering prescribed information in a developed view of a human-head shape by digitizing and/or coding a hair design according to the one that a customer requests, and treating or styling hair based on this developed view.

The present invention relates to a method for styling hair based on a three-dimensional human-head model, on whose respective regions, strips stating digitized and/or coded information of a hair design according to the one that a customer requests, are pasted.

The present invention relates to a developed view of a human-head shape and a three-dimensional human-head-shape model, on which strips stating digitized and/or coded information of a hair design according to the one that a customer requests are pasted. In the present invention, hair styling or hair treating implies all the treatment regarding hair styling which is done for beauty and/or hairdressing purposes to include hair cutting, hair coloring, hair ironing, etc.

‘A developed view of a human-head shape’ in the present invention implies a planar view that is prepared by laying out a three-dimensional human-head shape in two dimensions and dividing the planar view into prescribed regions, and ‘a three-dimensional human-head-shape model’ implies a three-dimensional model of a human head.

### BACKGROUND ART

Although styling hair, particularly permanent-waving hair, is very important for women, generally it is seldom that a hair style is successfully embodied only by permanent-waving hair. Instead, a beautiful hair style is normally achieved by combining a permanent wave with hair cutting, hair blowing, etc.

Thus, although female customers considered that designing of a hair style and styling of hair as designed were important factors for making a good impression of themselves on others, the reality they faced was a long way from designing hair cutting, hair coloring or ironing that accommodates their tastes by themselves or actualizing the hair style based on the design.

Conventionally in styling hair, a customer decides a hair style for hair cutting, hair coloring or a permanent wave that she likes to have by consulting photos of hair models, hair designs, magazines, or advice from a hairstylist, and then tells the hairstylist what she has decided. Based on such customer's order, the hairstylist does the hair; otherwise the hair style has no choice to be finished by relying totally on the hairstylist's memory on the customer's order. Additionally, because the hair styling that the customer wants is not recorded accurately on paper, it relies mostly on the hairstylist's sensibility. In reality, it has been common practice that a hairstylist is left in charge of how hair cutting, hair coloring or permanently waving is done, just suiting the occasion. In this way, regarding hair styling, conventionally there was no choice but to rely on a hairstylist's “intuition” and good sense, or a hairstylist's excellent techniques, if he/she would have those characteristics.

In this sense, there was room that so-called “charismatic” hairstylists could exist.

Thus, in the existing circumstances, beauty salons that female customers use are by and large selected to specific salons, and that hair styling of the customers is entrusted to specific hairstylists working at those beauty salons. Furthermore, it is difficult to exactly actualize a hair style as told by a customer. Even if the hair style that a customer liked to have could be actualized a single time, it was impossible to reproduce the same hair style the next time when the hair is styled.

In such a situation, designing of the hair style by a customer or hair styling based on the hair style designed did not completely satisfy the customer's request. Consequently, the hair had no choice to be styled case by case.

For example, if looking at the conventional hair styling techniques as for hair styling, it has become common for a customer to determine a design by pasting a model sheet of a hair design that the customer requested while receiving advice from a hairstylist (Japanese Utility Model Application Laid-open Nos. 1983-48180, 1984-98821, 1984-98822, 1985-64722, and 1993-91519). It was difficult, however, to actualize the hair style design exactly as requested by customers because of each customer having a different head-shape. Not to mention, it was more difficult to reproduce the hair style designed.

Additionally, it has become publicly known that to facilitate design changes made to a hair design requested by a customer, a design for a hair style accommodating the customer's taste is worked out by using predicted pattern images of the requested hair style produced on the TV monitor or the computer screen while comparing it with a hair style model (Japanese Patent Application Laid-open Nos. 1983-97306, 1981-109616, 1985-45303, 1995-67721, 1997-98834 and 1935-14655). However, when actually applying the three-dimensional visual information projected on the screen to hair styling, the results were short of what satisfied the customer's needs. Furthermore, use of a three-dimensional human-head-shape model on which a number of synthetic false hair (in some cases, actual human hair was used) is planted was proposed. In terms of space, storage and complexity, it was difficult to reuse the human-head-shape model.

As described above, if attempting to reproduce the same hair style as finished at the previous time, getting information on the same design had to depend on the same beauty salon or the same hairstylist. Moreover, the design information from the previous time was merely an outline of the image information of a human-head shape. Because detailed design information covering details of the human-head-shape model was not recorded accurately, it was difficult to finish hair styling that satisfied the customer. The results were the same even if using transferred data on computers between beauty salons under the same chain. Designing a hair style accommodating a customer's taste by the customer had never been given a thought.

### DISCLOSURE OF INVENTION

In the case of adults, on the entire head, each hair has a thickness of on the average 0.5 to 0.09 mm and a stretching rate of approximately 50%, and 50 to 100 hairs naturally fall out per day. On a regular basis, approximately 100,000 to 150,000 hairs exist on the head. Hair grows approximately 1.1 to 1.3 cm a month. Although cases depend on a female customer's genetic make-up, hair can be cut or permanent-waved every four to five weeks. At this time, even though the customer orders a hair style that she likes to have every time she gets her hair cut or permanent-waved, the result all depends on individual hairstylist's skills and it is quite common that the customer leaves the beauty salon somewhat discontent that the finished hair style has not been done as she wanted.

Thus, every hair design order that the customer requests differs, and hair styling tastes and technical skills of hairstylists, with which the customer's hair is styled, are of all descriptions. For these reasons, it is extremely difficult to find a perfect match of a design requested by a customer with a hairstylist's skills. In practice, it is a fact that actualizing the customer's order has to depend on each hairstylist's taste and techniques as described above.

Assuming that a hairstylist's techniques would be excellent and hair styling that a customer wants could be achieved, if the customer requests the same hair styling the next time when she visits the beauty salon, which most of customers normally do, and if the same hairstylist as the previous time would be available, the customer's request could be understood to some extent. Even with the same hairstylist available, because accurate data of the previous hair design was not recorded, the hairstylist has to rely on her/his memory about the previous hair styling. If it is a different hairstylist that is available, the customer's request is not understood completely.

This means that reproduction of the exact same hair styling of the previous hair cutting, hair coloring or permanent-wave was impossible. Still more, if a hairstylist was different from the one who did the hair the last time, reproduction of the hair styling that the customer requests was hopeless.

With this background, it has become necessary in the present invention to solve problems in complete designing of a hair style that the customer requests, hair styling including hair cutting, hair coloring or giving a permanent wave based on the hair design requested, and complete reproduction of the customer's request.

Generally, finishing a hair style is roughly divided into setting the hair styling using curlers and then blow-drying hair styling using beauty products such as a dryer and a brush. Setting hair styling using curlers is a method comprising rolling up hair with curlers when hair is wet, drying the hair with the curlers rolled, removing curlers from the hair, and finishing the entire hair styling. Blow-drying hair styling is a method comprising dividing hair into some portions, styling the entire hair style by portion one by one while blow-drying the portion with a dryer.

Focusing attention on use of dress patterns in costume design where the customers' orders are actualized and reproduced using the patterns, inventors of the present invention have enabled the customers' orders to be more completely actualized and to be completely reproduced by applying the same concept to the hair styling.

The present invention is basically characterized in the following composition:

A method for designing a hair style comprising, entering numerical values and/or codes, which correspond to a hair design requested by a customer, in respective multiple divided regions in a developed view of a human head, wherein the method is used for styling hair based on the numerical values and/or the codes entered.

The method for designing a hair style as mentioned in (1) above, which is characterized in that the numerical values and/or codes to be entered in the respective regions corresponding to the hair design requested by the customer represent hair length.

The method for designing a hair style as mentioned in (1) or (2) above, which is characterized in that the numerical values and/or codes to be entered in the respective regions corresponding to the hair design requested by the customer represent a permanent-wave type for the hair design.

The method for designing a hair style as mentioned in any one of (1) to (3) above, which is characterized in that the numerical values and/or codes to be entered in the respective regions corresponding to the hair design requested by the customer represent a hair color type for the hair design.

A method for styling hair, which is characterized in styling hair actually based on a hair style designed in any one of (1) to (4) above.

A developed view of a human-head shape, which is characterized in that the developed view is divided into multiple large regions and many small regions for entering numerical values and/or codes corresponding to a hair design requested by a customer.

The developed view of a human-head shape mentioned in (6) above, which is characterized in that the above-mentioned multiple large regions are created by parting the human-head shape in the middle and further dividing the large regions into top front region, back region, top-side region, under-side region, and nape region.

The developed view of a human-head shape mentioned in (6) or (7) above, which is characterized in that the above-mentioned multiple large regions are front region, top region, side region, and back region of the human-head shape.

The developed view of a human-head shape mentioned in any one of (6) to (8) above, which is characterized in that the above-mentioned small regions are created by subdividing the large regions created by roughly dividing the human-head shape appropriately into appropriate number of regions.

The developed view of a human-head shape mentioned in any one of (6) to (9) above, which is characterized in that the number of the above-mentioned regions is approximately 10 to 160.

The developed view of a human-head shape mentioned in any one of (6) to (10) above, which is characterized in that, for each region of the developed view, a strip on which hair length as designed is entered in a given numerical value and/or code according to a predetermined design, is pasted.

A method for styling hair, which is characterized in styling hair based on the developed view mentioned in any one of (6) to (11) above.

An actual three-dimensional head-shape model, which is characterized in that, for each region of the three-dimensional human-head-shape model, which corresponds to the developed view mentioned in any one of (6) to (10), a strip on which hair length as designed is entered in a given numerical value and/or code according to a predetermined design, is pasted.

A method for styling hair, which is characterized in treating hair based on the three-dimensional human-head-shape model mentioned in (13) above.

Additionally, in the scope of the present invention, selling the developed views of the human-head shape mentioned in (6) to (11) above at appropriate places such as beauty salons is included.

The methods according to the present invention have repetitiveness/continuity and satisfy long-standing requests made by many people and their social contributions are great.

The present invention, as mentioned above, is basically characterized in that: customers' orders for hair styles are digitized or coded; using the numerical values and/or codes, and a developed view of the human-head shape, a hair style design special to each customer is created. The customer's hair style is designed based on the developed view of the human-head shape and the hair is styled based on the developed view.

Additionally, the present invention is characterized in that a developed view of a human-head shape, in which numerical values and/or codes regarding a hair design are entered, and a three-dimensional human-head-shape model, which is used as a base of the developed view can be created.

The developed view of the above-mentioned human-head shape is created by developing the human-head shape two-dimensionally, roughly dividing the human-head shape in two dimensions, and further creating smaller regions by subdividing large regions. Hair cutting, hair coloring or a permanent wave that a customer requests, which is/are instructed by the customer verbally, is entered in respective regions of the developed view in numerical values and/or codes according the design instructed by the customer.

In the present invention, digitizing and/or coding the predetermined hair design means entering, for example, the hair length from the base of hair for hair cutting, a given hair color type for coloring or a permanent-wave type for giving a permanent wave.

A hair design requested by a customer can be designed by a hairstylist in her/his own way on the spot or from the customer's stated personal request by referring to hair models while receiving advice from a hairstylist, or a hair design that the customer likes is prepared by the customer beforehand.

In the present invention, regarding the number of roughly divided large regions in the developed view; any number can be taken. However, it is appropriate to divide the front of the human-head shape into, for example, 10 front regions by parting it in the middle to right and left, which comprises top front, back, top, under-side and nape regions, or four regions comprising front, top, side and back regions. For the number of subdivided regions from the roughly divided regions, any number can be taken. It is appropriate, however, to subdivide them into 10 to 160, preferably 60 to 80 regions.

The present invention is characterized in that a predetermined hair design is entered for each region divided into large and small regions on the developed view of the above-mentioned human-head shape by digitizing and/or coding hair cutting, hair coloring or a permanent wave. In the present invention, complete actualization and reproduction of an order for hair cutting, hair coloring or a permanent wave requested by a customer can become possible by using a developed view on which a hair style designed by the customer's order is entered in numerical values and/or codes.

In the present invention, the basics are to enter a given numerical value and/or code in each region in the developed view according to the above-mentioned hair style designed. Additionally, a numerical value and/or code can be entered in each region in the developed view according to the above-mentioned predetermined hair design. If storage space is permitted, it is possible to paste strips, on which numerical values and/or codes according to the predetermined design are entered, on an actual human-head-shape model. For materials to be used for the strips, plain paper, cardboard, plastic sheets on which writing necessary information is easy and which can be cut easily (the paper is cut into the length of an actual designed hair style), can be used without restrictions.

By storing developed views in which numerical values and/or codes are entered according to hair styles designed, developed views on which strips stating given numerical values and/or codes are pasted, or actual human-head-shape models on which the above-mentioned strips are pasted, detailed records of the previous hair styles designed can be kept. With these records, using recorded developed views of designs or three-dimensional models, actualizing customers' designs not only becomes possible, but styling the hair in the same way as the last time also becomes possible. Consequently, reproducing the hair styling can be ensured.

With the developed view on which numerical values and/or codes are entered used as a base, the needs for each customer and prescription of hair styling can be understood by standardizing or integrating them, and the completely same hair styling can be produced referring to the developed view stored, even by different hair stylists or beauty salons. Additionally, hairstylists can give advice to a customer's request for a hair design properly.

Furthermore, it is more realistic to do hair styling based on a developed view on which numerical values and/or codes regarding a hair design are entered and a three-dimensional human-head model, which is used for a base for the developed view.

### BRIEF DESCRIPTION OF FIGURES

Using figures, hair styling techniques according to the present invention are described by contrasting with conventional hair styling techniques.

FIGS. 1 to 3 show conventionally-used basic division of a human-head shape. FIG. 1 shows conventionally-used basic division of a human-head shape (side); FIG. 2 shows conventionally-used basic division of a human-head shape (back); FIG. 3 shows conventionally-used basic division of a human-head shape (front).

FIGS. 4 to 6 show conventionally-used basic permanent-wave aspects. FIG. 4 shows a conventional permanent-wave example (side); FIG. 5 shows a conventional permanent-wave example (back); FIG. 6 shows a conventional permanent-wave example (front).

FIG. 7 shows a hair growth degree seen from the front.

FIG. 8 shows a hair growth degree seen from the side.

FIGS. 9 to 11 show basic human-head-shape division according to the present invention. Particularly, FIG. 9 shows an example of a large region (side) of the human-head shape according to the present invention. FIG. 10 shows an example of large regions (back) of the human-head shape according to the present invention. FIG. 11 shows an example of large regions (front) of the human-head shape according to the present invention.

FIGS. 12 and 13 show the division shown in FIGS. 9 to 11 in developed views with each region divided shown.

FIGS. 14 to 16 show another example of a human-head shape according to the present invention, showing division seen from each side, the back and the front.

FIGS. 17 and 18 show developed views divided based on the regions shown in FIGS. 14 to 16 in the same manner as FIGS. 9 to 11.

FIGS. 19 and 20 show views in which the hair design according to the present invention is stylized in a human-head shape.

FIG. 21 shows an example of entering numerical values and/or codes in each region. An example for how to enter is shown in FIG. 21 and can be pasted using a strip on a human-head-shape model as shown in FIG. 19 or FIG. 20.

Codes shown in the figures are as follows:

LTF: Left Top Front

LTS: Left Top Side

LUS: Left Under-side

LNA: Left Nape

F: Front

T: Top

BC: Back center

SL: Side left

BL: Back left

SR: Side right

BR: Back right

### BEST MODES FOR CARRYING THE INVENTION

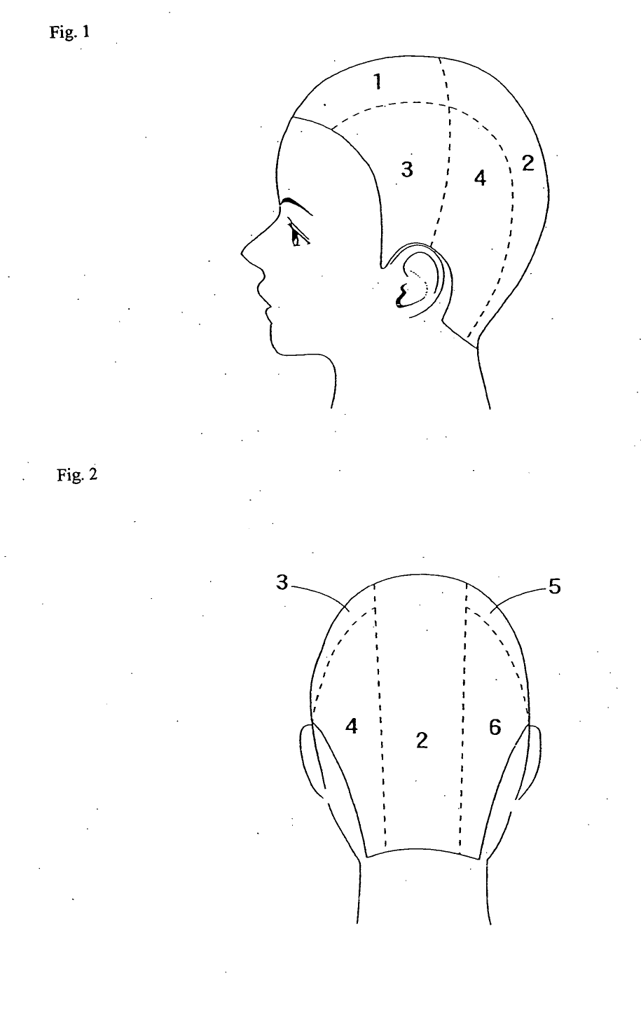

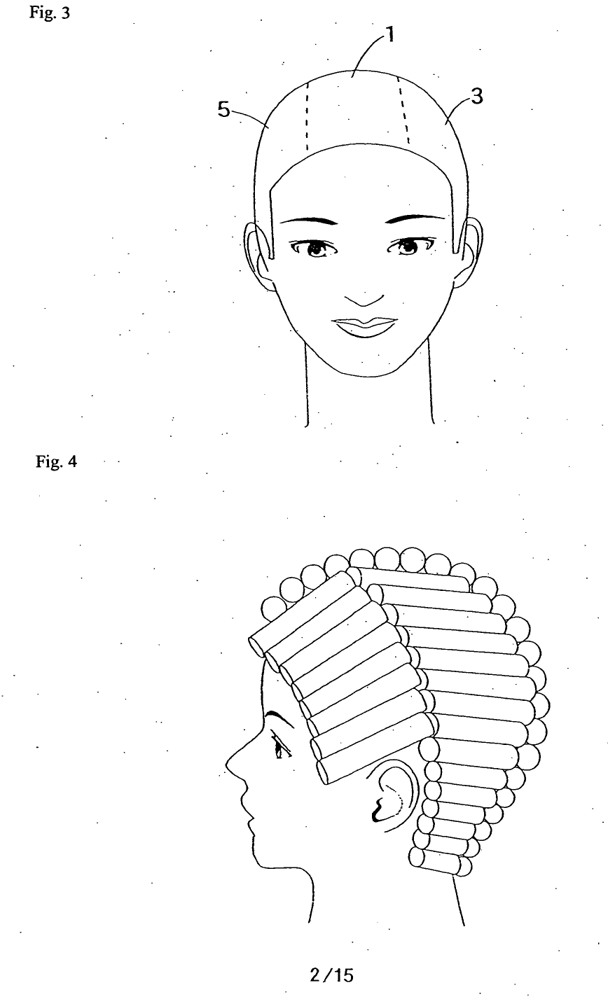

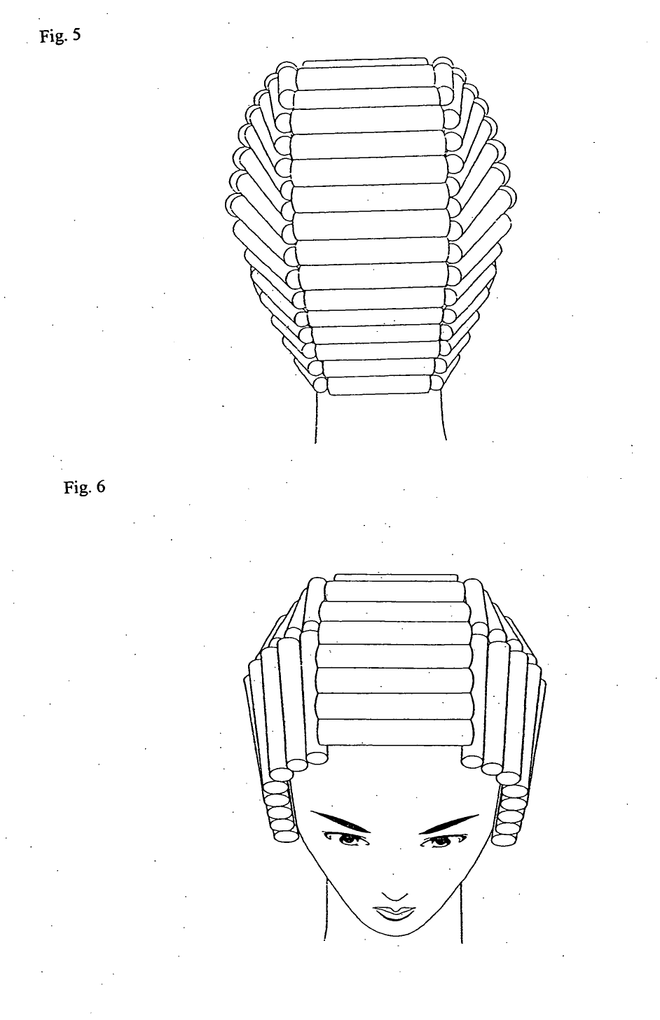

Conventionally, a human-head-shape model is roughly divided and subdivided. As for these divisions, as shown in FIGS. 1 to 3, it was common to divide it into approximately 1 to 6 regions ranging from the front to the back front, the left side and the right side. Conventionally, it was common practice to give a permanent-wave for each region as shown in FIGS. 4 to 6 according to the divisions shown in FIGS. 1 to 3. Additionally, although cutting and a permanent wave were done by listening to a customer's request for a hair design, a finished hair style was not always the same as that the customer requested.

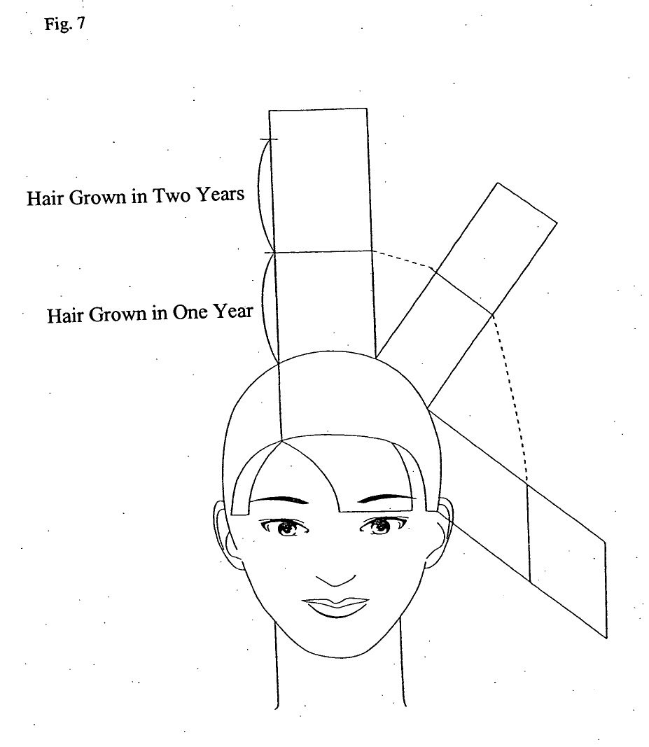

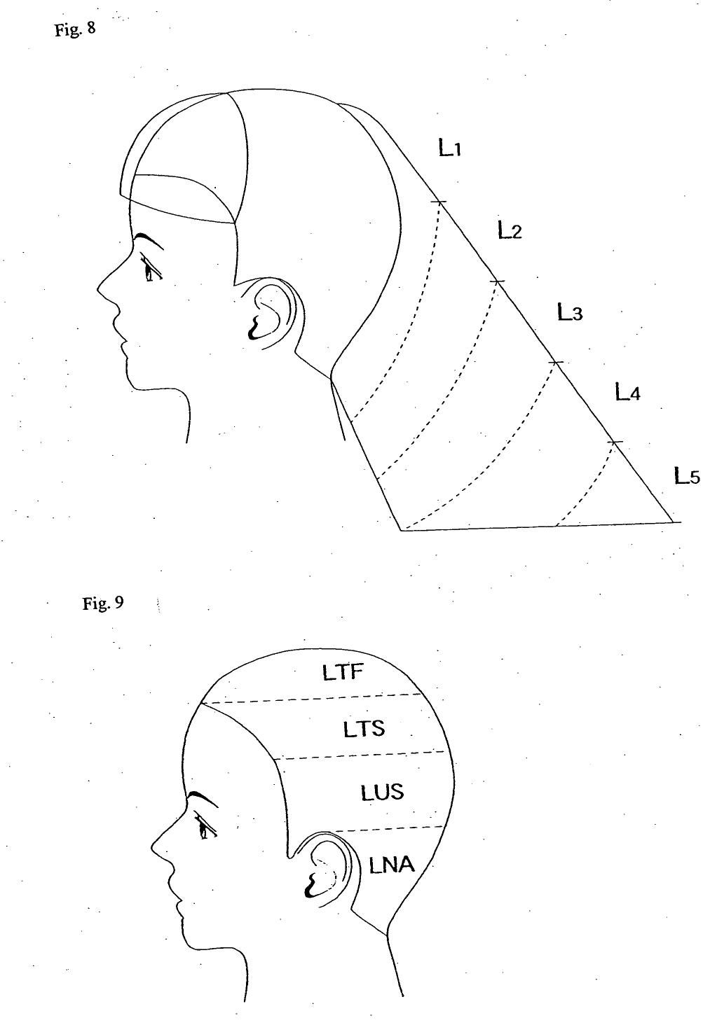

On the contrary, in the present invention, a style of hair for cutting, a permanent wave and hair coloring is designed, and hair ranging from hair grown in one year to hair grown in many years as shown in FIG. 7 and FIG. 8, respectively, is styled using a developed view of a human-head shape. Incidentally, L₁ in FIG. 8 implies hair grown in one year and L₂ implies hair grown in two years. L₃ and L₄ respectively imply hair grown in three and four years and L₅ implies hair grown in five years.

[Specifying Regions]

In the present invention, a human-head shape is, first, divided into four regions: the Top Front (TF), Top Side (TS), Under Side (US) and Nape (NA). Further, these regions are parted in the middle to the right and the left into a total of eight regions. At this time, the left-side seen from the rear of the head is the Left and the right-side seen from the rear of the head is the Right.

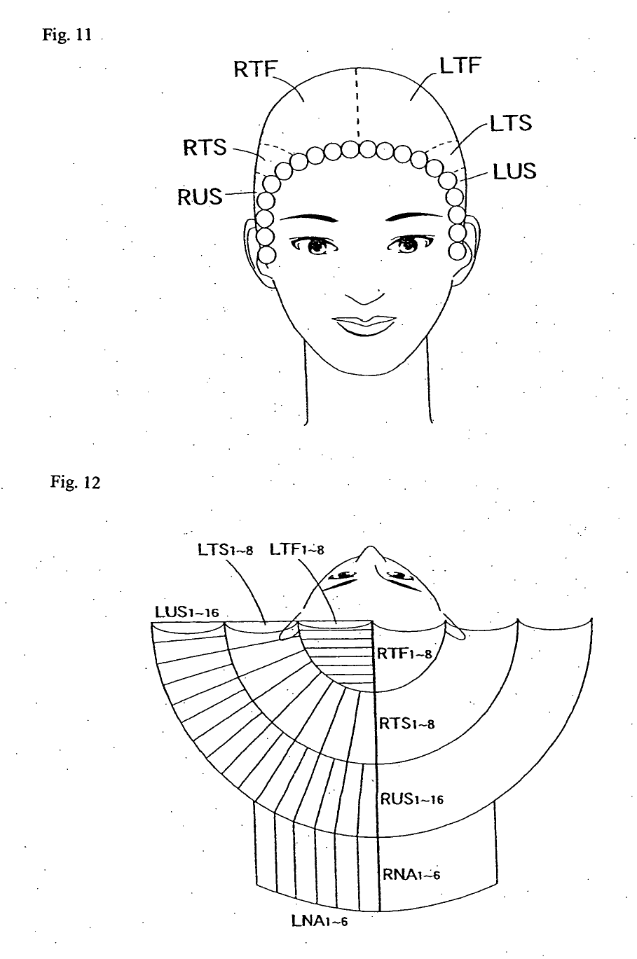

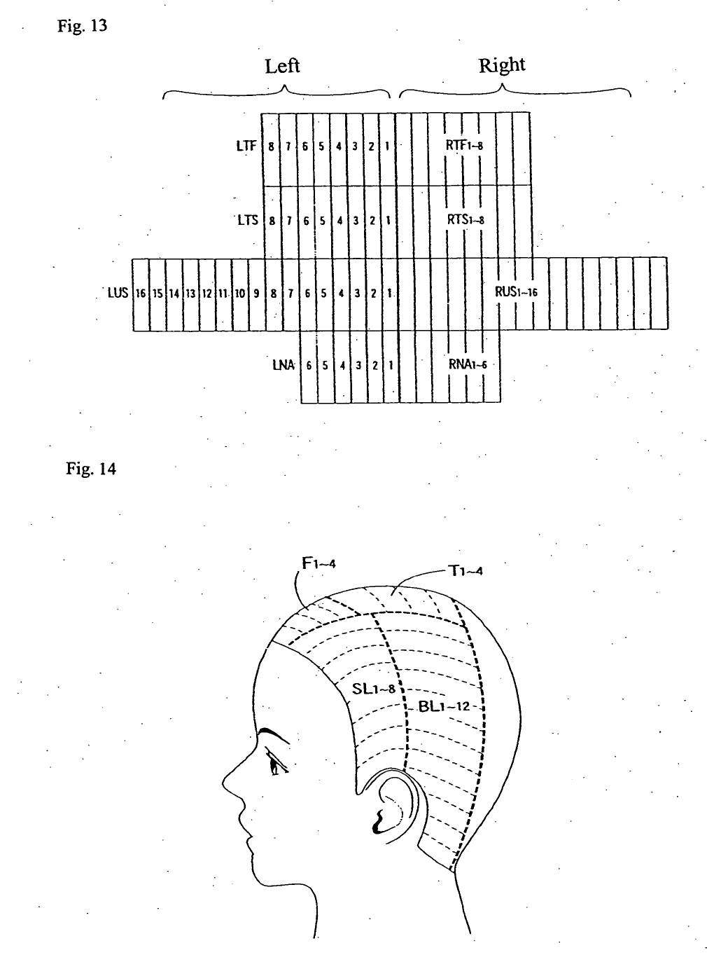

In the present invention, one unit, which is created by further subdividing the eight regions (4×2), is regarded as one region. By coding the regions using codes 1 to n, the position of each region is clarified. FIG. 12 shows these regions three-dimensionally and FIG. 13 shows the regions two-dimensionally.

In an example of the division according to the present invention, the Top Front (TF) is divided into eight, the Top Side (TS) is divided into eight, the Under Side (US) is divided into sixteen and the Nape (N) is divided into six, making a total of thirty-eight regions on either side. Thus, for the left and right sides, a total of seventy-six (38×2) units are created. In this division, for example, “LTS₅” shows a region on the left side of the back center line, and the fifth position from the top of the Top Side region in the large region LTS (Left Top Side) on the left side of the head shown in FIG. 9.

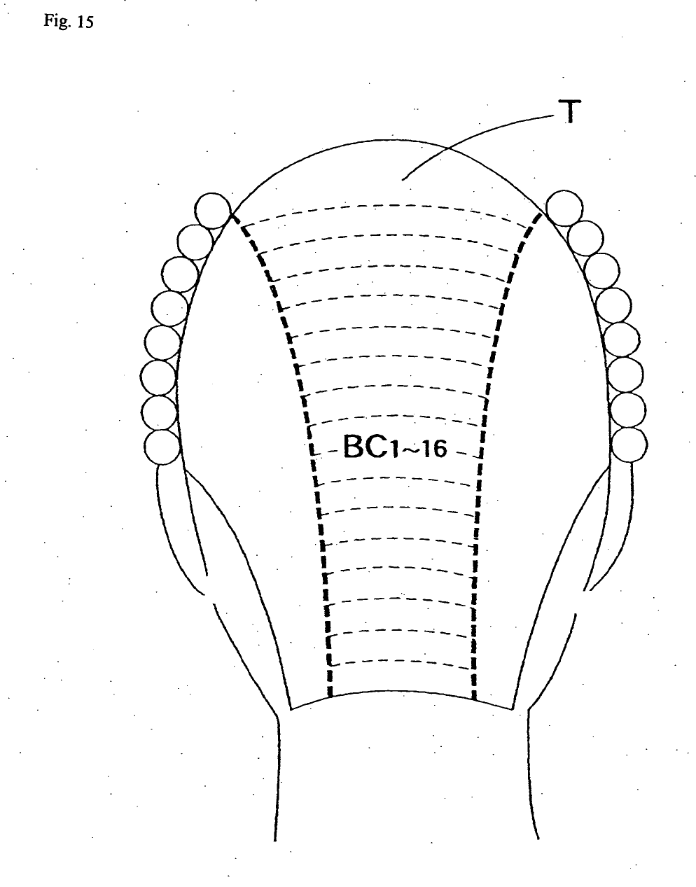

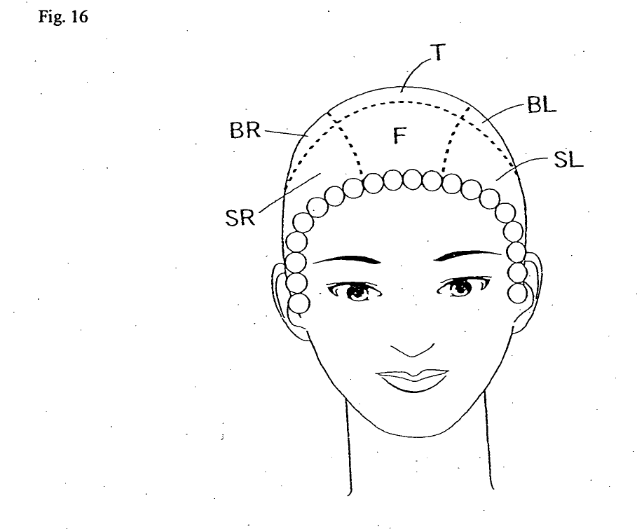

Depending on a hair style, there are cases where dividing a head-shape from the Top Front one by one and not providing the center of the head-shape may suit the purpose. In this case, as shown in FIGS. 14 to 16, it is possible to determine regions by roughly dividing the head shape into seven regions comprising the Front, Top, Back Center, Side Left, Back Left, Side Right and Back Right, and further subdividing them into small regions.

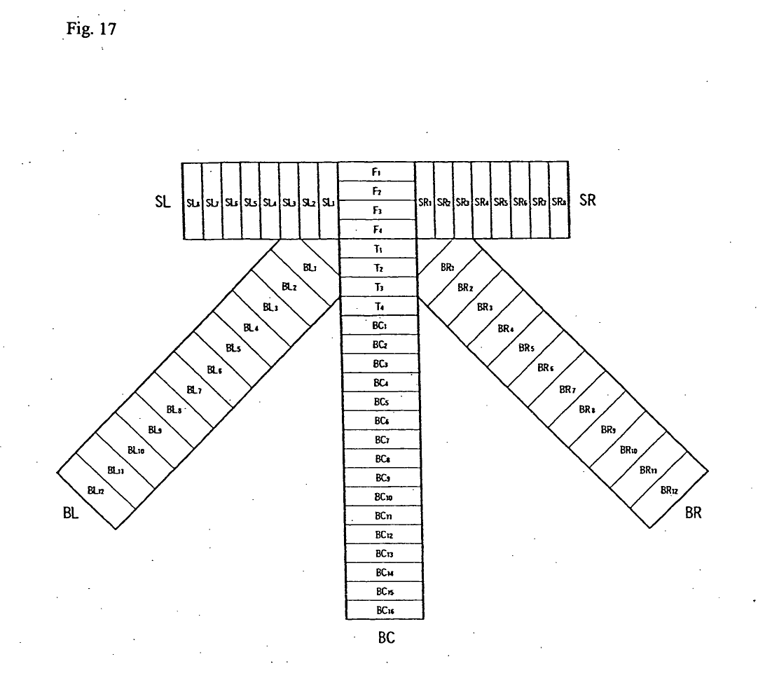

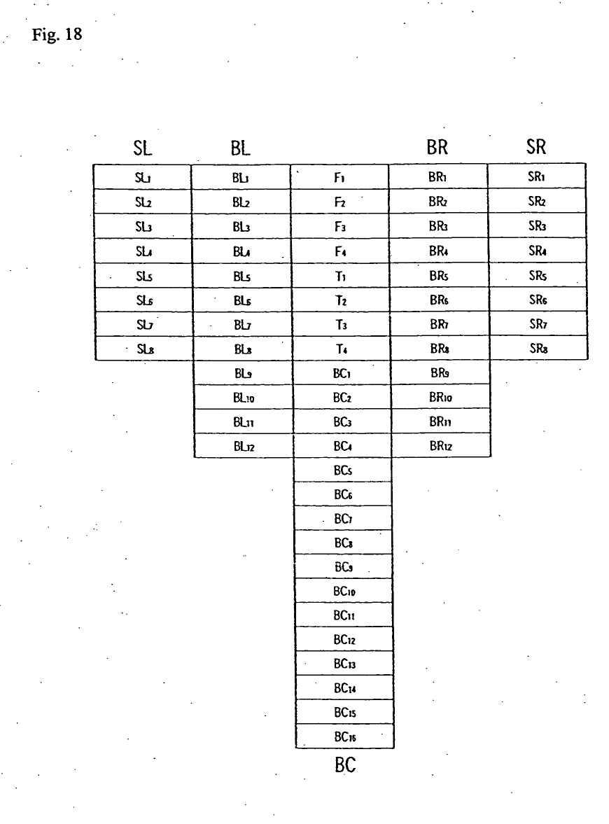

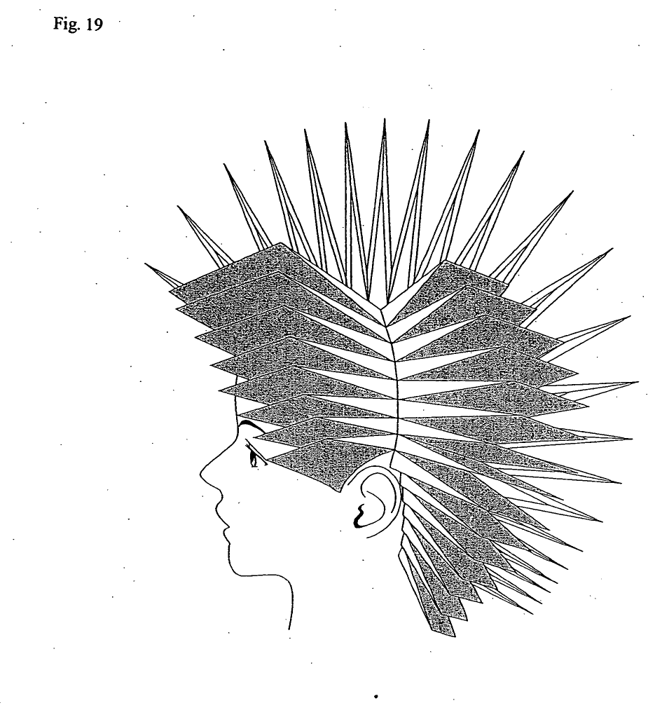

Consequently, in FIG. 17 or FIG. 18 which is developed views of the above-mentioned FIGS. 14 to 16 showing roughly divided regions, the Front (F) and the Top (T) are respectively divided into four regions, the Back Center (BC) is divided into sixteen, the Side Left (SL) and the Side Right (SR) are respectively divided into eight and the Back Left (BL) and the Back Right (BR) are respectively divided into twelve. In the case of this division, if referring to FIG. 14, for example, “BL₂” shows the third region from the top of the Back Left (BL) region seen from the back of the head-shape.

[Designing Hair Styles]

As shown in FIGS. 7 and 8, hair grows as indicated by hair grown in one year and hair grown in two years, and is normally cut appropriately by a hairstylist to the appropriate length and then styled.

In other words, a hair style that a customer requests is designed. To design a hair style, based on a photograph of a hair model, etc. and referring to timely advice given by a hairstylist, a desired hair design is determined by a customer. Based on this hair design, the hairstylist cuts hair by drawing from her/his intuition and permanent-waves hair so that the desired hair style of the customer, which was designed in advance using a model as shown in FIGS. 4 to 6, is to be finished. In the present invention, each region in the above-mentioned developed view corresponds to the desired hair design of the customer. It is the basics that the hair length, the length to be cut and a permanent-wave type or a hair color type in numerical values and/or codes are entered, and that a permanent-wave or coloring hair is done based on these numerical values or codes.

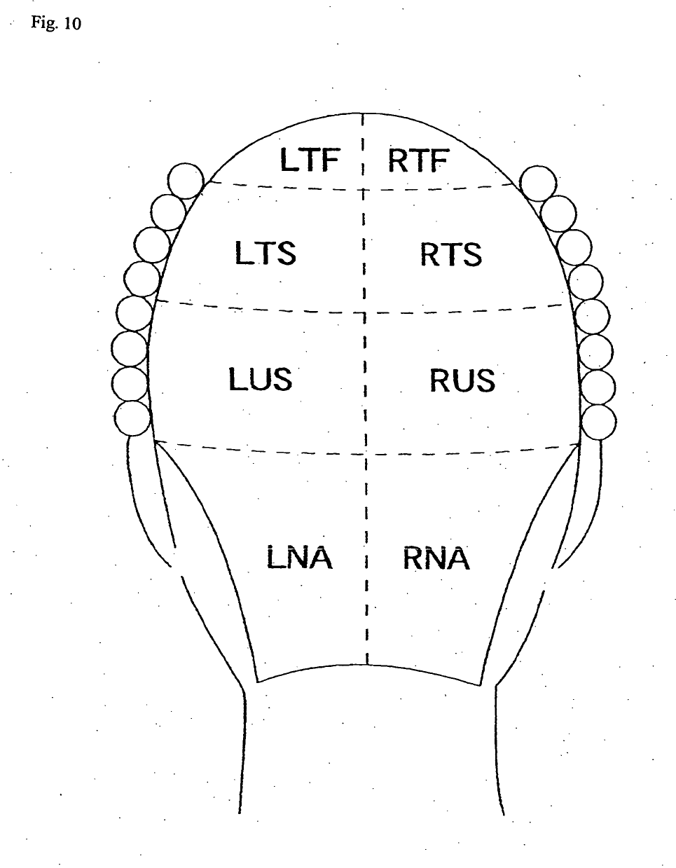

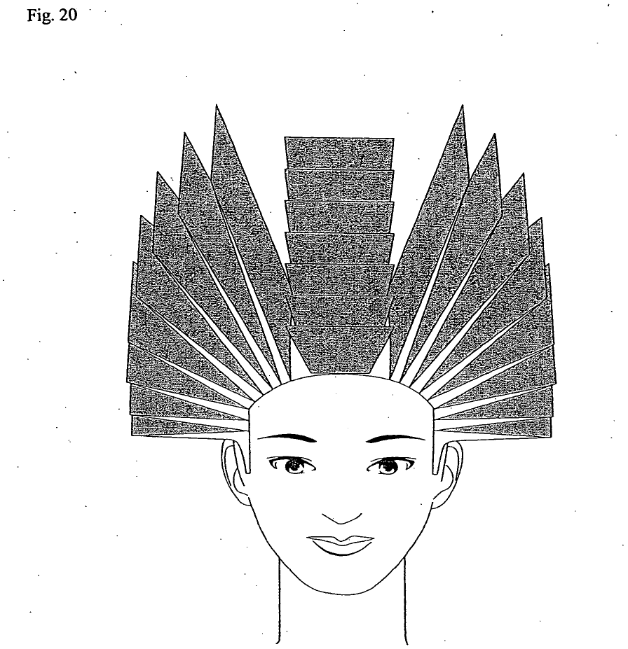

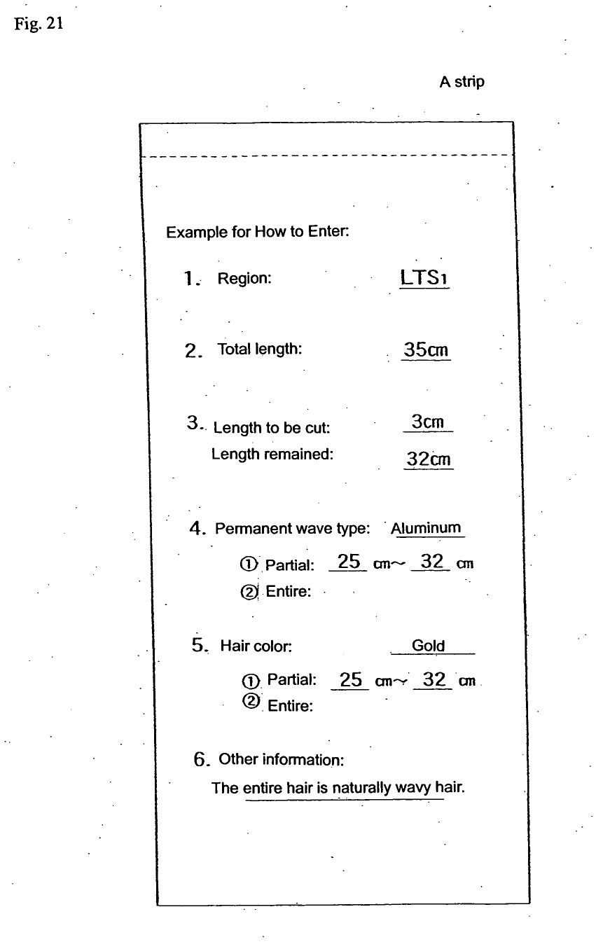

In the present invention, the hair length, the length to be cut and a permanent-wave type or a hair color type, those other items corresponding to a hair design that each customer requests are entered in numerical values and/or codes in respective regions developed from large regions of the above-mentioned human-head shapes. These are shown in FIGS. 9 to 11, in respective regions in FIGS. 17 and 18, which are developed from human-head shapes, or in FIGS. 14 to 16. Thus, it is easy to imagine the design from FIG. 12. However, it is the design from FIG. 13, which depicts entering given codes and/or numerical values based on the design, that is easy. Numerical values and/or codes can be entered in spaces provided in respective regions in a developed view. In some cases, by cutting out an entry table shown in FIG. 21 into strips, it is possible to paste the strips on respective regions in the developed view or on an actual three-dimensional human-head-shape model as shown in FIG. 19 or FIG. 20. In this case, the number of the strips corresponding to the number of the regions is required.

Regarding a hair design, it can be designed based on a hair model photo and referring to a hairstylist's advice as is done conventionally or, for the customer's convenience, for the customer herself to determine a design before actual styling of the hair and to enter numerical values and/or codes in developed views such as shown in FIGS. 12, 13, 17 or 18. For this reason, it is possible to sell sheets of a developed view that is divided into regions at beauty salons, etc., to make it more convenient for customers.

As mentioned above, by entering numerical values and/or codes in the developed view of a hair design according to the present invention, a hair design that a customer likes can be expressed completely and in detail. Then cutting, a permanent wave or hair coloring can easily be embodied. Additionally, because different hairstylists or distantly-located beauty salons can easily obtain the information on the customer's request regardless of lapse in time of hair styling between the previous time and the present time, and styling of the same design can surely be reproduced.

### INDUSTRIAL APPLICABILITY

As the result of the present invention, hair styling can be done by accurately actualizing a request made by a customer, as compared with conventional hair styling by hairstylists, barbers, etc. Additionally, because it is possible to store hair styles as data, hair styling can be done in a convincing way, and accurate reproduction is possible. Thus, exchange of hair design information using recorded data between different hairstylists and beauty salons, hair styling techniques can be standardized.

1. A method for designing a hair style comprising, entering numerical values and/or codes, which correspond to a hair design requested by a customer, in respective multiple divided regions in a developed view of a human head, wherein the method is used for styling hair based on the numerical values and/or the codes entered.

2. The method for designing a hair style according to claim 1, which is characterized in that said numerical values and/or codes to be entered in the respective regions corresponding to the hair design requested by the customer represent hair length.

3. The method for designing a hair style according to claim 1 or claim 2, which is characterized in that said numerical values and/or codes to be entered in the respective regions corresponding to the hair design requested by the customer represent a permanent-wave type for the hair design.

4. The method for designing a hair style according to any one of claims 1 to 3, which is characterized in that said numerical values and/or codes to be entered in the respective regions corresponding to the hair design requested by the customer represent a hair color type for the hair design.

5. A method for styling hair, which is characterized by styling hair based on the hair style designed in any one of claims 1 to 4.

6. A developed view of a human-head shape, which is characterized in that the developed view is divided into multiple large regions and many small regions for entering numerical values and/or codes corresponding to a hair design requested by a customer.

7. The developed view of a human-head shape according to claim 6, which is characterized in that said multiple large regions are created by parting the human-head shape in the middle and further dividing the large regions into top front region, back region, top-side region, under-side region, and nape region.

8. The developed view of a human-head shape according to claim 6 or claim 7, which is characterized in that said multiple large regions are front region, top region, side region, and back region of the human-head shape.

9. The developed view of a human-head shape according to any one of claims 6 to 8, which is characterized in that said many small regions are created by subdividing the large regions created by roughly dividing the human-head shape appropriately into appropriate number of regions.

10. The developed view of a human-head shape according to any one of claims 6 to 9, which is characterized in that the number of said regions is approximately 10 to 160.

11. The developed view of a human-head shape according to any one of claims 6 to 10, which is characterized in that, for each region of the developed view, a strip on which hair length as designed is entered in a given numerical value and/or code according to a predetermined design, is pasted.

12. A method for styling hair, which is characterized by styling hair based on the developed view claimed in any one of claims 6 to 11.

13. An actual three-dimensional head-shape model, which is characterized in that for each region of the three-dimensional human-head-shape model, which corresponds to the developed view claimed in any one of claims 6 to 10, a strip on which hair length as designed is entered in a given numerical value and/or code according to a predetermined design, is pasted.

14. A method for styling hair, which is characterized by styling hair based on the three-dimensional human-head-shape model claimed in claim 13.
